(19) United States
(12) Patent Application Publication
(10) Pub. No.: US 2012/0203210 A1
(43) Pub. Date: Aug. 9, 2012

(54) Urinary catheter and method

(76) Inventors: Richard W. Schanz, St. George, UT (US); J. Bryan Ellsworth, St. George, UT (US)

(21) Appl. No.: 12/931,657

(22) Filed: Feb. 7, 2011

(51) Int. Cl. A61M 27/00

(52) U.S. Cl. 604/544

(57) ABSTRACT

A flexible removable catheter with varying bell-shaped cross sectional widths for insertion through the urethra extending into the bladder to allow urine to flow through a urine passageway into a catheter bag.

### BACKGROUND OF THE INVENTION

1. Field of Invention

This invention pertains to urinary catheters. More particularly it relates to a flexible removable catheter for insertion through the urethra extending into the bladder to allow urine to flow through the urine passageway.

2. Description of Related Art

A number of catheters to relieve urine retention are known. A urinary catheter is a tube that can be inserted through the urethra into the bladder to allow drainage. The process of inserting a catheter involves in most uses, insertion of a rigid delivery tube carrying a thin, flexible tube catheter (“soft” catheter), which is left in the urethra for draining. In other designs a larger, solid (“hard”) catheter is inserted.

An individual who needs to use a catheter for a few days is not totally uncomfortable and is easily able to tolerate its uniqueness. However, an individual that has to use a catheter for a longer period of time finds that it is discomforting. The discomfort is in direct proportion to the daily activities of the wearer. A person whose daily activity is primarily walking around or standing has the least amount of problem. For a person who sits a great deal, such as an office worker, the catheter is truly a nuisance. If the person is a truck driver or an equipment operator where the vibrations transmit into the body especially when sitting through the seat of the pants, the catheter presents many problems. The firmness of the shaft of the typical catheter creates the sensation of sitting on a rod and will soon change from being very uncomfortable to inflammation and possibly resulting in infection.

Some of the catheters generally inserted with a positioning device are Eshel et al., U.S. Pat. No. 6,893,430 issued May 17, 2005 using a tubular guide; Bolmsjo et al, U.S. Pat. No. 7,662,145 issued Feb. 16, 2010 with insertion tool 22; and Hakky et al., U.S. Pat. No. 5,713,829 using applicator rod 12.

Others have a rigid insertion tip with an expanding ballooning segment to seal the catheter within the bladder as a Foley type of catheter. Kim, U.S. 2005/0124978 published Jun. 9, 2005 discloses an indwelling urinary catheter with a rigid insertion end, and an expandable segment to prevent urine discharge except through the catheter. Crisp, U.S. 2004/0199086 published Oct. 7, 2004 uses an elongated pliable tube for bladder insertion with an inflatable balloon positioned beneath a drain opening inserted into the bladder to prevent urine discharge except through the catheter. Christopher, U.S. Pat. No. 4,710,169 issued Dec. 1, 1987 discloses an indwelling urinary catheter inserted with a semi-rigid tubular portion entering the urethra with a ballooning end to prevent urine discharge except through the catheter. Dikeman et al., U.S. 2006/0095019 published May 4, 2006 discloses another urinary catheter with a flexible insertion end positioned beneath a drain opening using an inflatable balloon to prevent urine discharge except through the catheter. Boxer, U.S. Pat. No. 4,155,364 issued May 22, 1979 discloses another catheter assembly with a flexible filoform and a flexible follower detachably connected to the end of the filoform. This tip with a drain opening is positioned below an inflatable balloon, which similarly prevents urine discharge except through the catheter. McWhorter, U.S. Pat. No. 3,769,981 issued Nov. 6, 1973 is another urinary catheter with a ballooning positioning tip beneath a drain opening inserted into the bladder to prevent urine discharge except through the catheter.

Hakky et al., U.S. Pat. No. 6,096,013 issued Aug. 1, 2000 discloses a catheter with fluid injected to cause catheter wall stiffening for assistance during insertion. Rods 34 are then inserted to separate end sections 18 and 20 inside the bladder to properly drain urine through the catheter.

The invention described below provides a practical flexible catheter containing a removable spine to stiffen the catheter to aid in insertion. After insertion, the spine is then removed to provide a flexible catheter, which is more comfortable to wear, and avoids the need of a separate insertion device.

### SUMMARY OF THE INVENTION

The present invention comprises a catheter for placement within the urethra of a patient extending to the bladder to aid in urination. The catheter comprises a shaft of a length and diameter to be inserted within the urethra and extend to the bladder, with first and second ends. Running the length of the catheter is a liquid drain passageway for transporting urine with an inlet and outlet.

A spine passageway runs the length of the shaft with a closed end proximate the first end of the shaft and an open end proximate the second end of the shaft. An air/liquid inflation passageway with open first and second ends also runs the length of the shaft.

A ballooning securing tip is mounted proximate the first end of the shaft and is in communication with the first end of air/liquid inflation passageway. It is structured to either inflate/deflate when filled or emptied with air or liquid to secure the securing tip and first end of the shaft within the bladder. Thus positioned, the inlet of the liquid drain passageway is positioned within the bladder to drain urine when secured by expansion of the securing tip. Conversely, when the securing tip is contracted, it releases the ballooning securing tip for removal of the catheter.

A removable spine with a handle is positioned within the length of the spine passageways to stiffen the shaft to aid in its insertion within the urethra and bladder. The handle extends beyond the open end of the spine passageways for grasping to remove the spine after shaft insertion to allow the shaft to flex for patient comfort during use.

An inflation/deflation pump with an internal air/liquid reservoir is in communication with the inlet of the air/liquid passageway, which, when activated in a first mode, forces air/liquid from the air/liquid reservoir to inflate the expandable ballooning securing tip to hold the first end of the catheter within the bladder, and in a second mode deflates the ballooning securing tip to release the catheter for removal.

A catheter bag is then associated with the drain outlet of the liquid drain passageway to store urine drained from the bladder, or a plug wherein the bladder retains the urine until the patient removes the plug to drain the bladder. The patient then replaces the plug.

In one embodiment, the catheter has its inner and outer surfaces coated with silver and zinc to reduce infection. In another embodiment, the outer surfaces of the catheter are coated with an antibiotic impregnated silicone to reduce over growth of bacteria.

The catheter is preferably made of a flexible material resistant to bacterial adhesion. Typical flexible materials are silicone, rubber, latex, and thermoplastic elastomers. Silicone has the advantage of being inert and unreactive to body fluids.

The catheter is placed within a patient by employing the above catheter in the stiffened state with the spine in place to aid in positioning the first end of shaft within the urethra and bladder of the patient. When positioned, the spine is removed and the ballooning securing tip is inflated to secure the inlet of the liquid drain passageway within the bladder to collect and insure that urine is drained through the liquid drain passageway and out the drain outlet.

A bag is then affixed to the drain outlet to collect urine until the bag is changed, emptied, or plugged as discussed.

When there is no further need for the catheter, the ballooning securing tip is deflated and the catheter withdrawn and disposed of along with the bag.

The catheter thus provides a simple to use device, which has the placement advantages of a stiffened catheter, but the comfort of a flexible catheter. The catheter thus provides the advantages of hard and flexible catheters without the need for a placement guide or device.

### DESCRIPTION OF THE DRAWINGS

FIG. 1 is a cross-sectional side view of a conventional catheter.

FIG. 2 is a cross-sectional view of the catheter of FIG. 1.

FIG. 3 is a cross-sectional side view of a preferred embodiment of the invention; and

FIG. 4 is a cross-sectional view of the preferred embodiment of FIG. 3; and

FIG. 5 is a cross-sectional view of the preferred embodiment of FIG. 3 under compression.

### DESCRIPTION OF THE ILLUSTRATED EMBODIMENTS

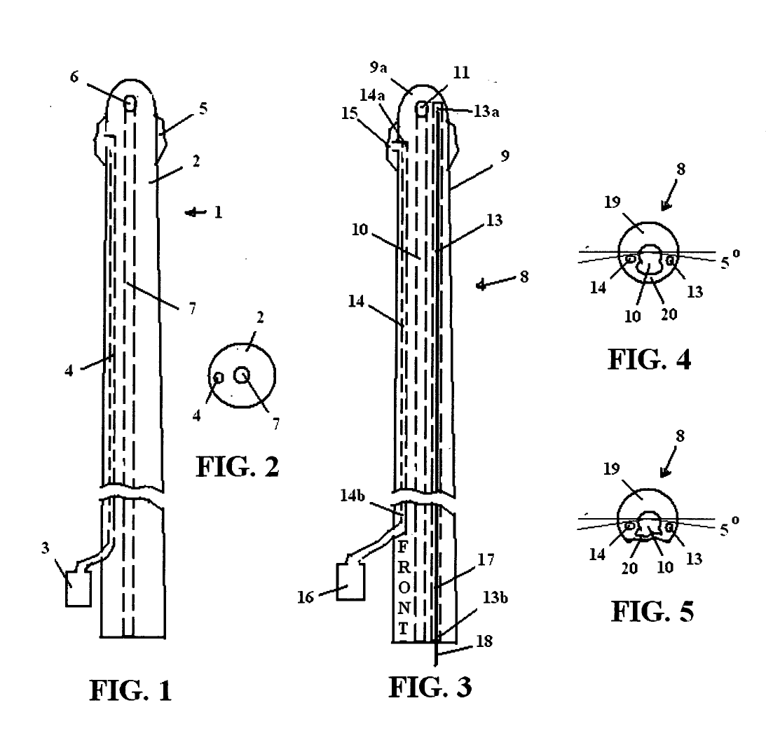

FIG. 1 is a cross-sectional side view of a conventional catheter 1 with unforgiving thick tubing 2. It uses an adapter 3 for injection of water through passageway 4 in communication with a balloon 5 attached to the tip of the tubing 2, which when inflated holds the catheter 1 in place. Urine flows through the opening 6 leading into a urine passageway 7 to a catheter bag (not shown). The sidewalls 2 of the catheter are firm so that the catheter can be inserted, causing discomfort over long use or sitting.

FIG. 2 is a cross sectional view of the catheter 1 of FIG. 1 showing that water passageway 4 and the urine passageway 7 are circular, and subject to pinching off when a user sits bending the catheter 1.

FIG. 3 is a cross-sectional side view of the present catheter 8. It has a shaft 9 of a length and diameter to be inserted within the urethra and extend into the bladder, with first and second ends 9a, 9b. The shaft 9 has a urine drain passageway 10 for transporting urine with an inlet 11 and outlet 12. This urine drain passageway 10 has a bell shaped cross sectional area shown in FIG. 4, which resists kinking when bent and compressed.

A spine passageway 13 runs the length of the shaft 9 with a closed end 13a proximate the first end 9a of the shaft 9 and an open end 13b proximate the second end 9b of the shaft 9.

An air/liquid inflation passageway 14 with first and second ends 14a, 14b runs the length of the shaft 9 to inflate a balloon securing tip 15.

The balloon securing tip 15 is mounted proximate the first end 9a of the shaft 9 and is in communication with the air/liquid inflation passageway 14. It is structured to either inflate/deflate when filled or emptied with air or liquid from an adapter 16 to secure the first end of the shaft 9a within the bladder. This positions the inlet 11 of the urine drain passageway 10 within the bladder to drain urine and prevents leakage when expanded. When contracted by the adapter 16, the balloon securing tip 15 releases for removal of the catheter 8.

A removable spine 17 with a handle 18 is positioned within the spine passageways 13 to stiffen the shaft 9 to aid in insertion within the urethra and bladder. The handle 18 extends beyond the open end 13b of the spine passageway 13 and is structured for grasping after shaft insertion to aid in removal of the spine 17. Removal of the spine 17 after placement allows the catheter 8 shaft 9 to flex and bend when in place.

The adapter 16 is an inflation/deflation pump with an internal liquid reservoir in communication with the inlet of the air/liquid inflation passageway 14. When activated in a first mode, the adapter 16 forces liquid from a liquid reservoir to inflate the expandable balloon securing tip 15 to hold the first end 9a of the catheter 8 within the bladder, and in a second mode deflates the balloon securing tip 14 to release the catheter 8 for removal.

The end 12 of the urine drain passageway 10 is associated with a catheter bag (not shown) to collect urine. The catheter bag is emptied, discarded, or replaced when necessary.

The spine 17 consists of a thin wire or plastic line and travels the full length of the catheter through spine passageway 17a. The spine 17 provides stability and sufficient rigidity during insertion to make sure the catheter 8 is properly positioned. When the catheter 8 is properly in place, the spine 17 is removed by pulling on the spine handle 18 and discarded.

FIG. 4 shows how the top half of the catheter 8 positions the air/liquid inflation passageway 14 and the spine passageway 13 five degrees below the centerline of the catheter 8 shaft 9 on opposite sides of the bell shaped urine drain passageway 10. The thickness of the shaft 9 upper wall 19 between the air/liquid inflation passageway 14 and the spine passageway 13 is approximately 1.6 mm, while the thickness of the shaft 9 lower wall 20 is 0.6 mm. This allows the urine passageway 10 to remain open when the shaft 9 is compressed by sitting as shown in FIG. 5.

The overall outside diameter of the catheter is 7.2 mm to 8.0 mm. The length of the upper segment of the catheter 8 shaft 9 is 102 mm entering the bladder segment with an inner and outer wall 19, 20 diameter of approximately 1.6 mm. At this point, the compression effect on the catheter 8 is encountered requiring a compression segment employing a thin wall 20 of 0.6 mm, which continues from 102 to 153 mm at which point the catheter 8 returns to the normal 1.6 mm wall diameter.

The bell-shaped cross sectional design of the compression segment of the catheter 8 accommodates the weight of a user, when sitting, and allows the urinary passageway 10 to partially compress/collapse without cutting off urine flow. This eliminates the feeling the user may have of sitting on a rod, resulting is comfort. Normal shifting of the user's body allows the urine passageway 10 to open to its non compressed state so that any clots or foreign matter may flow through in a normal manner.

In one embodiment, the word “FRONT” is stamped on the catheter 8 at its lower end indicating the catheter 8 is properly installed when the word “FRONT” is pointed away from a patient's body.

Although this specification has referred to the specific embodiments, it is not intended to restrict the scope of the claims. The scope of the invention is, therefore, indicated by the appended claims, rather than by the foregoing description. All changes that come within the meaning and range of equivalency of the claims are to be embraced within their scope.

We claim:

1. A catheter for placing within the urethra of a patient extending to the bladder to aid in urination comprising:
a. a flexible shaft with walls of a length and diameter to be inserted within the urethra and extend to the bladder, with first and second ends defining
i. a urine drain passageway for transporting urine with an inlet and outlet,
ii. at least one spine passageway running the length of the shaft with a closed end proximate the first end of the shaft and an open end proximate the second end of the shaft, and
iii. an air/liquid inflation passageway with first and second ends running the length of the shaft,
b. a balloon securing tip mounted proximate the first end of the shaft in communication with the air/liquid passageway structured to either inflate/deflate when filled or emptied with air or liquid to secure the first end of the shaft within the bladder to position the inlet of the urine drain passageway within the bladder to drain urine when expanded, and to contract and release the balloon securing tip for removal of the catheter,
c. a removable spine with removal end of a length and size to be positioned within the spine passageway to stiffen the shaft to aid in insertion within the urethra and bladder, and the removal end extending beyond the spine passageway open end for grasping after shaft insertion for removal of the spine to allow the shaft to flex in place, and
d. an inflation/deflation pump with an internal liquid reservoir in communication with the inlet of the air/liquid passageway, which, when activated in a first mode, forces liquid from the liquid reservoir to inflate the expandable balloon securing tip to hold the first end of the catheter within the bladder, and in a second mode deflates the balloon securing tip to release the catheter for removal; the urine drain passageway outlet adapted to attach and empty into a catheter bag.

2. A catheter according to claim 1, wherein inner and outer surfaces of the catheter are coated with silver and zinc to reduce infection.

3. A catheter according to claim 1, wherein outer surfaces of the catheter are coated with an antibiotic impregnated silicone to reduce over growth of tissue.

4. A catheter according to claim 1, wherein the catheter is constructed of a material resistant to bacterial adhesion.

5. A catheter according to claim 1, wherein the cross-sectional area of the urine drain passageway is bell shaped to prevent kinking when a user fitted with the catheter sits.

6. A catheter according to claim 5, wherein the center of the cross section of the urine drain passageway is off-set from center five degrees and is bell shaped.

7. A catheter according to claim 6, wherein the catheter has three different segments with differing cross sectional widths, an upper insertion segment, which is thicker than a second compression segment which allows the catheter passageway to partially compress without obstruction when a user sites, and a third lower segment with a cross sectional width approximately the width of the upper insertion segment.

8. A method for placing a catheter within a patient comprising:
I. employing a catheter for placement within the urethra and bladder of the patient having:
a. a shaft of a length and diameter to be inserted within the urethra and extend to the bladder, with first and second ends defining
i. a liquid drain passageway for transporting urine with an inlet and outlet,
ii. at least one spine passageway running the length of the shaft with an open end proximate the first end of the shaft and a closed end proximate the second end of the shaft, and
iii. an air/liquid passageway with first and second ends running the length of the shaft,
b. a ballooning securing tip mounted proximate the first end of the shaft in communication with the air/liquid passageway structured to either inflate/deflate when filled or emptied with air or liquid to expand and secure the first end of the shaft within the bladder and position the inlet of the liquid drain passageway within the bladder to drain urine when expanded, and to contract and release the ballooning securing tip for removal of the catheter,
c. a removable spine with removal ends of a length to be positioned within the spine passageway to stiffen the shaft to aid in insertion within the urethra and bladder, and extend the removal ends beyond the spine passageway open ends for grasping after shaft insertion for removal of the spines to allow the shaft to flex,
d. an inflation/deflation pump with an internal liquid reservoir in communication with the inlet of the air/liquid passageway, which, when activated in a first mode, forces liquid from the liquid reservoir to inflate the expandable balloon securing tip to hold the first end of the catheter within the bladder, and in a second mode deflates the ballooning securing to release the catheter for removal,
II. attaching a catheter bag to the liquid drain passageway outlet to collect urine in the catheter bag, and
III. removing the catheter bag and catheter after use.
